(19) United States
(12) Patent Application Publication
(10) Pub. No.: US 2026/0320365 A1
(43) Pub. Date: Oct. 15, 2026

(54) SYSTEMS AND METHODS OF SCALABLE CONVEX RECURRENT NEURAL NETWORK TRAINING FOR INTERPRETING BRAIN DYNAMICS

(71) Applicants: NTT Research, Inc., Sunnyvale, CA (US); President and Fellows of Harvard College, Cambridge, MA (US); The Board of Trustees of the Leland Stanford Junior University, Stanford, CA (US)

(72) Inventors: Fatih DINC, Stanford, CA (US); Hidenori TANAKA, Belmont, MA (US)

(73) Assignees: NTT Research, Inc., Sunnyvale, CA (US); President and Fellows of Harvard College, Cambridge, MA (US); The Board of Trustees of the Leland Stanford Junior University, Stanford, CA (US)

(21) Appl. No.: 19/484,569

(22) PCT Filed: May 20, 2024
(86) PCT No.: PCT/US2024/030221

§ 371 Date: Nov. 14, 2025

Related U.S. Application Data
(60) Provisional application No. 63/503,324, filed on May 19, 2023.

(51) Int. Cl. G06N 3/06

(52) U.S. Cl. CPC G06N 3/061

(57) ABSTRACT

Computer-implemented methods and systems for training a neural network to interpret neural dynamics, the method comprising obtaining functional connectivity between brain regions and artificial neurons simulated by the neural network, capturing neural activities between biological neurons, processing the captured neural activities to obtain training data for the neural network, and constructing the neural network using the training data to reproduce neural dynamics between the artificial neurons.

### CROSS-REFERENCE TO RELATED APPLICATIONS/PRIORITY

The present invention claims priority to U.S. Provisional Patent Application No. 63/503,324 filed May 19, 2023, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### BACKGROUND

Understanding how the brain works is a complex problem that researchers so far have attacked from various angles. Despite the many beneficial applications of such understanding, current technologies fail for many reasons. There remains a real and pressing a need for the networks to scale to 10000s of neurons and be able to learn and predict in real-time support of brain-machine interfaces.

### SUMMARY

Wherefore, it is an object of some embodiments of the presently disclosed invention to overcome some or all of above-mentioned shortcomings and drawbacks associated with the current technology.

The presently disclosed invention relates to computer-implemented methods for training a neural network to interpret neural dynamics, the method comprising obtaining functional connectivity between brain regions and artificial neurons simulated by the neural network, capturing neural activities between biological neurons, processing the captured neural activities to obtain training data for the neural network, and constructing the neural network using the training data to reproduce neural dynamics between the artificial neurons. In some embodiments the neural network includes a convex solver. In some embodiments the convex solver comprises a convex weighted loss function. In some embodiments, computer-implemented method further comprises optimizing a loss of the convex solver via a fixed-point estimation. In some embodiments, computer-implemented method further comprises predicting a spatial and temporal activity of neural population dynamics. In some embodiments the neural network is configured to extract large-scale functional connectivity between brain regions and/or simulated networks. In some embodiments the neural network is trained to predict the temporal activity of a corresponding biological neuron in the brain. In some embodiments the neural network is configured to uncover attractor structures within the brain dynamics. In some embodiments the neural network is further configured to design external interventions to control neural population dynamics in the brain. In some embodiments the neural network is trained to interpret neural dynamics in real-time to support brain-machine interfaces. In some embodiments wherein the convex solver is further configured to perform optimization updates accelerated on a graphics processing unit (GPU). In some embodiments the convex solver is part of an alternating direction method of multipliers (ADMM) framework. In some embodiments the convex solver is configured to enforce constraints on the neural network during training. In some embodiments the constraints include preventing self-excitation of neurons within the neural network. In some embodiments the convex solver is configured to utilize biological priors for inductive biases during training. In some embodiments the convex solver is configured to handle input noise and conversion noise during the training of the neural network. In some embodiments the convex solver is configured to perform outlier detection during the training process. In some embodiments the convex solver is configured to scale to datasets collected from more than 10,000 neurons for offline processing.

The presently disclosed invention further relates to non-transitory computer-readable mediums comprising instructions that, when executed by one or more processors, cause a computer system to obtain functional connectivity between brain regions and artificial neurons simulated by the neural network, capture neural activities between biological neurons, process the captured neural activities to obtain training data for the neural network, and construct the neural network using the training data to reproduce neural dynamics between the artificial neurons.

The presently disclosed invention further relates to a computer system for training a neural network to interpret neural dynamics, the system comprising one or more processors and a non-transitory computer-readable medium including instructions that, when executed by the one or more processors, cause the computer system to obtain functional connectivity between brain regions and artificial neurons simulated by the neural network, capture neural activities between biological neurons, process the captured neural activities to obtain training data for the neural network, and construct the neural network using the training data to reproduce neural dynamics between the artificial neurons. In some embodiments the neural network is a data constrained recurrent neural network.

The presently disclosed invention is directed to methods and apparatuses systems and methods that relate to a scalable convex recurrent neural network trained for interpreting brain dynamics are described herein. These systems and methods allow extraction of large-scale functional connectivity between brain regions and/or simulated networks. Embodiments of the present disclosure may include a computer-implemented method for training a neural network to interpret neural dynamics, including obtaining functional connectivity between brain regions and artificial neurons simulated by the neural network, including capturing neural activities between biological neurons. Embodiments may also include processing the neural activities between biological neurons to obtain training data for the neural network.

Embodiments may also include constructing the neural network using the training data. In some embodiments, the neural network may include a convex solver. Embodiments may also include reproducing neural dynamics between the artificial neurons simulated from the neural network. In some embodiments, the neural network may be a data constrained recurrent neural network.

In some embodiments, the convex solver includes a convex weighted loss function. In some embodiments, the method may include optimizing a loss of the convex solver via a fixed-point estimation. In some embodiments, the method may include predicting a spatial and temporal activity of neural population dynamics.

In some embodiments, the convex solver includes a convex weighted loss function. In some embodiments, the instructions, when executed, cause the one or more processors to further perform optimizing a loss of the convex solver via a fixed-point estimation. In some embodiments, the instructions, when executed, cause the one or more processors to further perform including predicting a spatial and temporal activity of neural population dynamics.

Embodiments of the present disclosure may also include a non-transitory computer readable medium including instructions to cause the one or more processors to perform obtaining a functional connectivity between brain regions and artificial neurons simulated by the neural network, including capturing neural activities between biological neurons. Embodiments may also include processing the neural activities between biological neurons to obtain training data for the neural network.

Embodiments may also include constructing the neural network using the training data. In some embodiments, the neural network may include a convex solver. Embodiments may also include reproducing neural dynamics between the artificial neurons simulated from the neural network. In some embodiments, the neural network may be a data constrained recurrent neural network.

Embodiments of the present disclosure may also include a computer system for training a neural network to interpret neural dynamics, including one or more processors. Embodiments may also include a non-transitory computer readable medium including instructions, when executed by the one or more processors, causing the computer system, to obtain a functional connectivity between brain regions and artificial neurons simulated by the neural network, including capturing neural activities between biological neurons.

Embodiments may also include processing the neural activities between biological neurons to obtain training data for the neural network. Embodiments may also include constructing the neural network using the training data. In some embodiments, the neural network may include a convex solver. Embodiments may also include reproducing neural dynamics between the artificial neurons simulated from the neural network. In some embodiments, the neural network may be a data constrained recurrent neural network.

In some embodiments, the convex solver includes a convex weighted loss function. In some embodiments, the instructions, when executed, cause the one or more processors to further perform optimizing a loss of the convex solver via a fixed-point estimation.

In some embodiments, the instructions, when executed, cause the one or more processors to further perform the step of predicting a spatial and temporal activity of neural population dynamics.

The foregoing summary is illustrative only and is not intended to be in any way limiting. Features from any of the disclosed embodiments can be used in combination with one another, without limitation. In addition to the illustrative aspects, embodiments, and features described above, further aspects, embodiments, and features will become apparent by reference to the drawings and the following detailed description.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 is a schematic representation showing interpreting and controlling brain dynamics with data constrained recurrent neural networks (dRNNs). The inventors present a large-scale functional connectivity task for artificial recurrent neural networks (RNNs), where each artificial neuron is trained to predict the temporal activity of a corresponding biological neuron in the brain. By extracting RNNs with functional connectivity, the inventors aim to achieve three goals in future work: (i) predict neural population dynamics, (ii) uncover attractor structures, and (iii) design external interventions to control neural population dynamics in the brain.

FIG. 2 is a schematic representation showing CoRNN: Convex and scalable solver for dRNNs via ADMM framework. The flowchart illustrates their CoRNN algorithm, which optimizes the parameters of a recurrent neural network to match the activity of a hidden unit with that of a generator (brain). The loss landscape is convex due to their careful choice of objective function. The algorithm starts by finding a fixed point for initialization and computing the inverse of the Hessian. It then starts a loop that goes through a certain number of iterations. At each iteration, the algorithm predicts the objectives, calculates the prediction error, updates the parameter values, and checks for convergence. The final output is the optimized parameters, which represent the functional connections between the neurons in the recurrent neural network.

FIGS. 3A and 3B are two graphs showing CoRNN achieves the best combination of speed and accuracy, dramatically accelerating convergence. The plot illustrates the relationship between accuracy (correlation between ground truth and inferred weights) and training time, measured in seconds on a log scale. The blue line represents the performance of their newly developed method, which combines fixed-point initialization and CoRNN, and achieves high accuracy while significantly accelerating convergence compared to other methods. Parameters: FIG. 3A: α=0.1, n_{rec}=200, T=3000, ϵ^{conv}~N (0, 10⁻⁴), ϵ^{input}~N (0, 10⁻²) and FIG. 3B: α=0.1, n_{rec}=200, T=3000, ϵ^{conv}~N (0, 10⁻⁵), ϵ^{input}~N (0, 10⁻¹). For both, error bars are standard error of the mean (s.e.m.) over 10 randomly initialized networks.

FIGS. 4A and 4B are two graphs showing CoRNN is much more stable and tolerant to variations in hyperparameter choices than FORCE. This plot describes the relationship between accuracy (correlation between ground truth and inferred weights) and the strength of regularization applied to the weights during training. The results show that while the FORCE algorithm, as previously applied to a similar task, is highly sensitive to the choice of hyperparameters, CoRNN maintains high accuracy across scales regardless of the strength of regularization. FIG. 4A with fixed point initialization for all and FIG. 4B with random initialization for Pytorch models and FORCE, whereas CoRNN is fixed-point initialized to define comparable scales. Parameters are the same as FIG. 3A. Error bars are sem.

FIG. 5 is a graph that shows ablating the fixed-point initialization degrades the convergence speed of the resulting models. The inventors conducted an ablation study to investigate the effectiveness of their proposed fixed-point initialization method. The results show that the CoRNN algorithm with fixed-point initialization (blue) outperforms all other methods, including the vanilla CoRNN without this fixed-point initialization technique. Parameters: α=0.1, n_{rec}=500, T=10000, ϵ^{conv}~N (0, 10⁻⁵), ϵ^{input}~N (0, 10⁻¹). Error bars are sem.

FIGS. 6A-6C are experimental results showing CoRNN can reproduce an RNN performing 3-bit flip flop task through dRNN training. The inventors use CoRNN to reproduce the behavior of a synthetic RNN trained on a k-bit flip-flop task, where the network is given a short input pulse at a random time. If the input pulse is +1, the output of the network must transition to +1 or stay at +1. If the input pulse is −1, the network's output must transition to −1 or stay at −1, ignoring input pulses that do not match. Through dRNN training, CoRNN is able to accurately reproduce (FIG. 6A) the inputs and outputs of the task-trained RNN, (FIG. 6B) spatiotemporal pattern of the neural population dynamics, and (FIG. 6C) the inputs and recurrent weights of the synthetic RNN. Parameters: α=0.9, n_{rec}=1000, T=200 trials (100 data points per trial), ϵ^{conv}~N (0, 10⁻³), ϵ^{input}~N (0, 10⁻²).

FIGS. 7A and 7B are two graphs showing Increasing Trial Data is Necessary for Learning from Larger Networks. FIG. 7A shows reconstruction accuracy of network outputs, measured as the correlation between the output units, and FIG. 7B shows reconstruction accuracy of network weights, measured as the correlation between recurrent weights, are plotted as a function of dataset size (number of trials) averaged over three networks. The different colors in the plot correspond to different number of neurons in the generator. Parameters: α=0.9, ϵ^{conv}~N (0, 10⁻³), ϵ^{input}~N (0, 10⁻²).

FIG. 8 is two plots showing CoRNN can scale to the largescale datasets.

Both CPU and GPU implementation of CoRNN are fast and scale faster than data generation process itself. Parameters: α=0.9, ϵ^{conv}~N (0, 10⁻³), ϵ^{input}~N (0, 10⁻²).

FIG. 9 is a block diagram illustrating a computing device, according to some embodiments of the present disclosure.

FIG. 10 is a flowchart illustrating a computer-implemented method for training a neural network, according to some embodiments of the present disclosure.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The terms “comprise(s),” “include(s),” “having,” “has,” “can,” “contain(s),” and grammatical equivalents and variants thereof, as used herein, are intended to be open-ended transitional phrases, terms, or words that do not preclude the possibility of additional acts or structures. are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. The singular forms “a,” “and” and “the” include plural references unless the context clearly dictates otherwise. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)−(a second number),” this means a range whose lower limit is the first number and whose upper limit is the second number. For example, 25 to 100 mm means a range whose lower limit is 25 mm, and whose upper limit is 100 mm.

The embodiments set forth the below represent the necessary information to enable those skilled in the art to practice the invention and illustrate the best mode of practicing the invention. For the measurements listed, embodiments including measurements plus or minus the measurement times 5%, 10%, 20%, 50% and 75% are also contemplated. For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “substantially” means that the property is within 80% of its desired value. In other embodiments, “substantially” means that the property is within 90% of its desired value. In other embodiments, “substantially” means that the property is within 95% of its desired value. In other embodiments, “substantially” means that the property is within 99% of its desired value. For example, the term “substantially complete” means that a process is at least 80% complete, for example. In other embodiments, the term “substantially complete” means that a process is at least 90% complete, for example. In other embodiments, the term “substantially complete” means that a process is at least 95% complete, for example. In other embodiments, the term “substantially complete” means that a process is at least 99% complete, for example.

The term “substantially” includes a value that is within 10% less than or greater than the indicated value. In certain embodiments, the value is within 5% less than or greater than of the indicated value. In certain embodiments, the value is within 2.5% less than or greater than of the indicated value. In certain embodiments, the value is within 1% less than or greater than of the indicated value. In certain embodiments, the value is within 0.5% less than or greater than of the indicated value.

The term “about” includes when value is within 10% of the indicated value. In certain embodiments, the value is within 5% of the indicated value. In certain embodiments, the value is within 2.5% of the indicated value. In certain embodiments, the value is within 1% of the indicated value. In certain embodiments, the value is within 0.5% of the indicated value.

In addition, the invention does not require that all the advantageous features and all the advantages of any of the embodiments need to be incorporated into every embodiment of the invention.

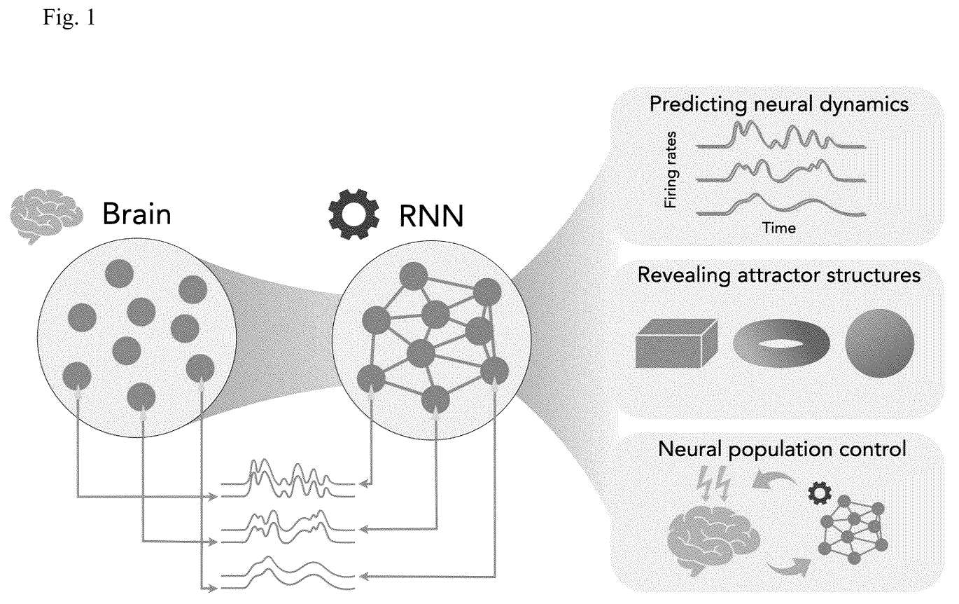

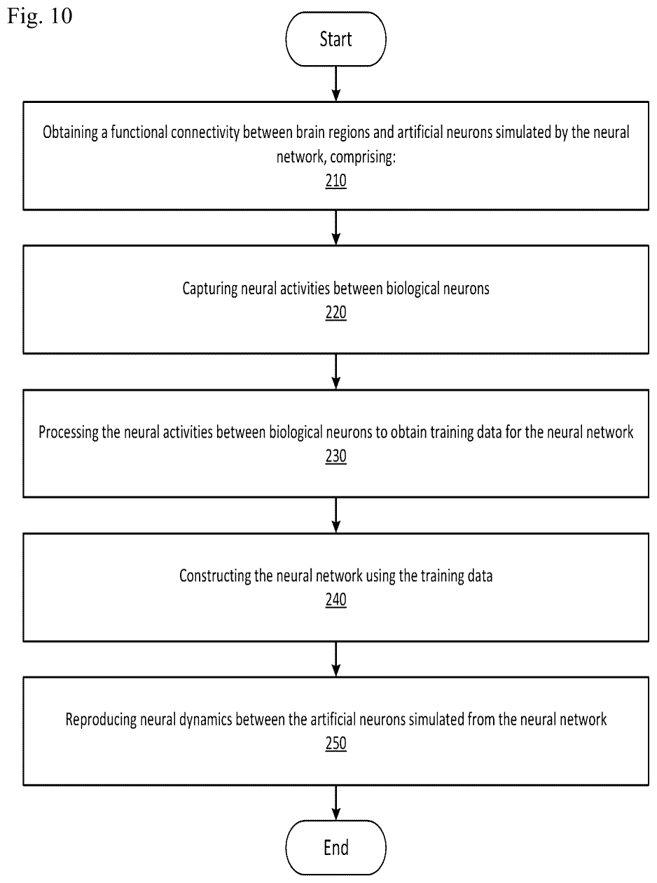

Turning now to FIGS. 1-12, a brief description concerning the various components of the present invention will now be briefly discussed.

Experimental neuroscience is advancing rapidly. As this progress continues, it becomes increasingly important to update theoretical and computational tools to keep pace. With the recent advances in targeted stimulation of individual neurons, biologically interpretable computational models with generative power are needed to supply these experiments with real-time computational control.

Among those biologically interpretable models are the data-constrained recurrent neural networks (dRNNs), where the number of units in the artificial network is matched to the number of neurons recorded, and the RNNs are trained to match the observed neural activities. The previous work in this paradigm uses FORCE learning, which was originally developed as an alternative to the reservoir computing paradigm for training read-out weights to produce a target function and then later applied to training recurrent weights to create dynamic attractors.

Data-constrained RNNs have immense potential to generalize the decades old approaches of estimating functional connectivity between brain regions through co-fluctuations that are still being utilized in systems neuroscience to allow reverse engineering underlying computations, to provide real-time computational control for causal experimentation, and to serve as a computational complement to the widely used optogenetics. Influencing biological experiments computationally on the fly requires models that can be trained and provide predictions in real-time. Additionally, biological interpretability of the underlying architecture is necessary for reverse-engineering the underlying computation. These requirements render many traditional machine learning approaches trained in large clusters with billions of parameters impractical for a direct application to experiments performed with limited computational resources, a large portion of which would be allocated to other computational procedures such as real-time extraction of neural signals.

When developing optimization procedures for data-constrained RNNs, another area of particular interest is the scalability of the training to data collected from several days and from more than 10,000 neurons for offline processing. With a model having scalable and fast learning capabilities, the inventors could potentially process multi-day imaging datasets and/or the brain-wide large datasets of the future and test biological hypothesis regarding large-scale computations at an instance. Although seven years passed since the original attempt at reproducing neural activities with data-constrained interpretable RNNs, the ability of the data constrained RNN training paradigm to influence biological/experimental progress has been limited if any. Perhaps one major contribution to this is the incompatibility of the FORCE approach with the realities of the experimental data, as well as its extremely low speed of convergence. In fact, the dRNN training has never been truly validated via a comparative study of potential optimizers. Moreover, previous work attempted to solve two problems at once, e.g. designing an optimizer for the data constrained RNN training paradigm and transforming the experimental neural activities to a format trainable by a vanilla recurrent neural network with sigmoid nonlinearity. Yet, both problems are interdisciplinary in nature and require full attention of their own. Thus, for the scope of this work, their focus is explicitly on the former, i.e. the validation of the data-constrained RNN training. The inventors leave the latter problem, e.g. the transformation of the experimentally observed neural activities into a suitable data format, to future work. In this disclosure, the inventors first present a task called “large-scale functional connectivity extraction,” in which the inventors attempt to learn the functional connectivity between “recorded neurons” simulated from a generator model. Then, the inventors introduce a convex and scalable solver, called CoRNN, and validate its accuracy and speed with respect to alternatives including FORCE employed by the seminal works. Contributions include the following: •The inventors introduce the large-scale functional connectivity extraction task and validate reproduction ability of data constrained RNNs on ground truth simulations •The inventors introduce a new convex solver called CoRNN: Scalable, stable, and interpretable convex recurrent neural networks for copying brain dynamics. •The inventors introduce a fixed-point initialization strategy to speed up convergence not only for CoRNN but for FORCE as well.

Unlike the traditional setting of dynamical system reconstruction task, where powerful algorithms (with potentially hidden units and deep layers) are trained to reproduce the underlying dynamical systems of an observation, the data-constrained RNN training aims to match the artificial units with the number of observed neurons to interpret currents shared between brain regions and/or population of neurons as proxy for functional connectivity. While ignoring the hidden units may seem too restrictive and confounding when reproducing observed neural activities, several critical biological observations motivate the use of dRNNs: 1) the observed neural activities tend to lie on low dimensional manifolds, 2) the noise correlations limit the maximum amount of information in the population, which can be recovered with few thousands of neurons, and 3) the currents shared between brain regions and/or simulated networks of networks are robust to sub sampling of individual neurons. In fact, instead of viewing dRNNs as performing a more restricted version of dynamical system reconstruction task, a more biologically relevant view would be to consider dRNNs as a generalization of functional connectivity estimation, which was originally performed via considering co-fluctuations between brain regions.

2. Theory 2.1. Data constrained RNN training for large scale functional connectivity extraction task the inventors start by introducing the large-scale functional connectivity extraction task, as shown in FIG. 1, which aims to reproduce interactions between brain regions and/or groups of neurons (e.g. functional connectivity). The inventors assume that the observations of real data can be captured by a generator RNN model. Then, the goal of the dRNNs is to reproduce the internal dynamics of the generator RNN. In most general case, observed neurons can be subsampled, or the genera tor might follow a different statistical model than dRNN, in which case the connectivity matrix learned by dRNN would not necessarily coincide with the underlying synaptic connectivity; rather be functional in essence.

For the scope of this work, the inventors aim to provide validity evidence for the optimization problem; thus, focus on the case with perfectly matching generator and learners. In this case, the terms functional and synaptical connectivity coincide, allowing a simple interpretation/scoring of the reproduction quality. The inventors test not only the accuracy of optimizers, but also focus on real-time learning and control possibility, robust ness to hyperparameters, scalability to whole brain. In its essence, large scale functional connectivity task is a compromise between co-fluctuations looked traditionally through noise and/or canonical correlations between brain regions; and the dynamical system reconstruction task, which traditionally uses more accurate/powerful approaches.

2.2. Generator model and estimator family of interest Following the trend in neuroscience, the inventors choose to focus on a recurrent neural network-based generator model. Specifically, the generators are assumed to be of the form:

$$τ \frac{{dr}_{i}}{dt} = - {r}_{i} + f ( {z}_{i} ) + {ϵ}_{i}^{con} , \qquad ( 1 )$$

$${z}_{i} = {\sum }_{j - 1}^{{N}_{rec}} {W}_{ij}^{rec} {r}_{j} + {\sum }_{j - 1}^{{N}_{in}} {W}_{ij}^{in} {u}_{j} + {ϵ}_{i}^{input} ,$$

where zᵢ is the total input to the recurrent unit i, rᵢ is the firing rate of the unit i, and f(.) is a pre-defined non-linearity,

$${W}_{ij}^{rec}$$

is the weight matrix for the recurrent connections,

$${W}_{ij}^{in}$$

is the weight matrix from the input to the recurrent units, u is the input vector, and

$${ϵ}_{i}^{input}$$

is the input noise to the recurrent units. Unlike previous literature, given their goal of reconstruction, the inventors also consider the existence of a conversion noise

$${ϵ}_{i}^{con}$$

that accounts for errors and/or miss-matches in the non-linearity. Additionally, the generator model has the constraint

$${W}_{ii}^{rec} = 0$$

preventing any self-excitation of neurons.

Suitable estimators for reconstructing such a model can be obtained (recursively) by discretizing the differential equation:

\hat{r}ₜ₊1,i=(1−α)\hat{r}_{t,i}+αf(\hat{z}_{t,i}) (2)

where the time scale parameter is defined as

$$α = \frac{Δ t}{τ} .$$

Here, the model parameters influence \hat{z}_{i,t} linearly such that

$${\hat{z}}_{t , i} = {\sum }_{j} {\hat{x}}_{t , j} {\hat{θ}}_{ji} = X \hat{θ} , \qquad ( 3 )$$

where we define the short-hand notations x=[r;u] and \hat{θ}=[W^{rec}; Wⁱⁿ]^{T} as concatenated matrices. The goal of the estimation problem is to find \hat{θ} such that \hat{θ}≈θ.

2.3 Convexification of the loss. Given that the overall problem is a regression problem, a reasonable choice would be to minimize the L2 loss:

$${\hat{ℒ}}_{2} ( \hat{θ} ) = {\sum }_{i = 1}^{{n}_{rec}} {\sum }_{t = 1}^{T} {( {\hat{r}}_{t , i} - {r}_{t , i} )}^{2} . \qquad ( 4 )$$

However, owing to the time recurrent definition of the general estimator \hat{r}_{t,i}, this minimization problem would require backpropagation through time. This approach would likely miss one of the inventors' main goals: being able to scale well to tens of thousands of neurons and hours of recordings on a personal computer.

As a first step, the inventors consider a smaller subset of estimators during training where \hat{r}_{t-1,i} is teacher-forced to r_{t-1,i} when computing \hat{r}_{t,i}. This turns the potentially infinite time problem into a single time-step one, which are called “single step prediction error paradigm”. Under this condition, the L2 error transforms to a simpler form:

$${ℒ}_{2} ( \hat{θ} ) = {α}^{2} {\sum }_{i = 1}^{{n}_{rec}} {\sum }_{t = 1}^{T} {( {\hat{d}}_{t , i} - {d}_{t , i} )}^{2} , \qquad ( 5 )$$

where we define

$${d}_{t , i} = \frac{rt + 1 , i - ( 1 - α ) rt , i}{α}$$

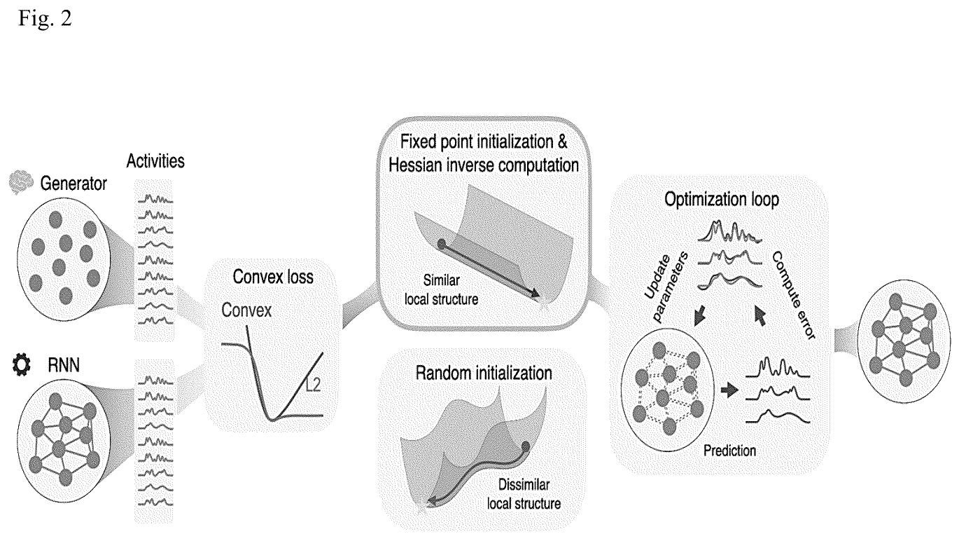

and correspondingly \hat{d}_{t,i}=f(\hat{z}) becomes an estimator consisting of a linear term through a single non-linearity. However, this simplified version of the loss is not convex and leads to a counter-intuitive global loss function with vanishing gradients in extremely large errors, as shown in FIG. 2.

Instead of minimizing the naive L2 loss, owing to the observation that

$$\frac{1 + \text{?}}{2}$$

would follow a linear+sigmoid form, the inventors replace the L2 loss with a cross-entropy loss that is well known to be convex under this estimator, see logistic regression. On the other hand, instead of a naive replacement, the inventors use a weighted loss of the form

$${ℒ}_{weighted} ( \hat{θ} ) = {\sum }_{i = 1}^{{n}_{rec}} {\sum }_{t = 1}^{T} {c}_{t , i} CE ( \frac{1 + {\hat{d}}_{t , i}}{2} , \frac{1 + {d}_{t , i}}{2} ) , \qquad ( 6 )$$

$$C ( a , b ) = - b log ( a ) - ( 1 - b ) log ( 1 - a ) .$$

Here, the inventors use CE to denote the cross-entropy loss and c_{t,i} are weighting factors chosen as

$${c}_{t i} = {[ 1 - {d}_{t , i}^{2} ]}^{- 1}$$

for the CoRNN solver for scalability as discussed below. For now, the regularized general loss function is written as

$${ℒ}_{CoRNN} ( \hat{θ} ) = \frac{1}{T} {ℒ}_{weighted} ( \hat{θ} ) + \frac{λ}{2} {‖ \hat{θ} ‖}_{F}^{2} , \qquad ( 7 )$$

where ∥\hat{θ}∥_{F} is the Frobenius norm of \hat{θ}.

2.4. Loss minimization with a scalable solver. Having convexified the loss function, the inventors next observe that the loss function itself is perfectly separable such that L_{CoRNN} (\hat{θ})=Σᵢ Lᵢ (\hat{θ}:,ᵢ). Thus, without loss of generality and for any given i, the subproblems concerning each scalar outputs dₜ:=d_{t,i} and corresponding (vector, not matrix) parameters \hat{β}:=\hat{β}(i)=\hat{θ}ⱼᵢ and cₜ:=c_{t,i} can be considered separately (See Appendix A) The gradient and Hessian of this smaller problem can be found as:

$${( {∇}_{\hat{β}} {ℒ}_{i} ( \hat{β} ) )}_{k} = \frac{1}{T} {\sum }_{t = 1}^{T} {c}_{t} [ {\hat{d}}_{t} - {d}_{t} ] {x}_{t , k} + λ {\hat{β}}_{k} , \qquad ( 8 a )$$

$${( {∇}_{\hat{β}}^{2} {ℒ}_{i} ( \hat{β} ) )}_{kl} = \frac{1}{T} {\sum }_{t = 1}^{T} {c}_{t} ( 1 - {\hat{d}}_{t}^{2} ) {x}_{t , k} {x}_{t , l} + {λδ}_{kl} \qquad ( 8 b )$$

In this naive form, the Hessian would need to be computed separately for each subproblem and after each parameter update as it depends on \hat{d}ₜ. This motivates the choice of

$${c}_{t} = {[ 1 - {d}_{t}^{2} ]}^{- 1} ,$$

since near the solution \hat{d}ₜ≈dₜ, the Hessian approximates to:

$$\begin{matrix}{( {∇}_{\hat{β}}^{2} {ℒ}_{i} ( \hat{β} ) )}_{kl} \approx \frac{1}{T} {\sum }_{t = 1}^{T} {x}_{t , k} {x}_{t , l} + {λδ}_{kl} \\ = \frac{1}{T} {X}^{T} X + λ I = {H}_{fp} ,\end{matrix} \qquad ( 9 )$$

which is both parameter \hat{β} (and thus \hat{θ}) and target dₜ independent; this is commonly shared across all subproblems as long as the convex program starts and remains nearby the optimum. Owing to its fixed nature, this Hessian is defined as the fixed-point Hessian and use this globally to perform optimization updates. Finally, the gradient of the full problem can be obtained by promoting Eq. 8a to a matrix:

$$\begin{matrix}{[ {∇}_{\hat{θ}} {ℒ}_{CoRNN} ( \hat{θ} ) ]}_{ji} = - \frac{1}{T} {\sum }_{t = 1}^{T} {\underbrace{\frac{[ {d}_{t , i} - {\hat{d}}_{t , i} ]}{1 - {d}_{t , i}^{2}}}}_{{E}_{t , i}} {x}_{t , j} + λ {\hat{θ}}_{ji} , \\ = - \frac{1}{T} {X}^{T} E + λ \hat{θ} ,\end{matrix} \qquad ( 10 )$$

where we define the prediction error matrix E. It is worth noting that each column of the gradient matrix contains the gradient for the scalar subproblem corresponding to the ith output. Then, defining the inverse (fixed-point) Hessian matrix as

A⁺=[X^{T}X+TλI]⁻¹. (11)
we obtain the following update rule (See Appendix A)

θ^{k+1}=A⁺X^{T}Xθ^{k}+A⁺X^{T}E^{k}, (12)
where all quantities that can be pre-computing the prediction error followed by matrix multiplications are underlined; this is well suited to be accelerated on a graphics processing unit (GPU).

2.5 Choosing a suitable fixed point. The approximation performed at the local Hessian stage, e.g.

$$\frac{1 - {d}_{t}^{- 2}}{1 - {d}_{t}^{2}} \approx 1 ,$$

requires that we initialize the parameters θ⁽⁰⁾:=θ_{fp} close to ≈θ*, where θ* is the solution to the optimization problem. If this condition is met, the approximate local Hessian derived in Eq. 9 would describe the local topology near the solution, the fixed point, and the steps in-between. As the inventors show in Appendix A, a suitable fixed-point candidate is the solution to the least squares problem on the currents Z:

θₗₛ=A⁺X^{T}Z. (13)

This step is computationally negligible, as the underlined matrix is already pre-computed for the iterations that follow.

3. Results. 3.1 Convex recurrent neural networks (CoRNN) via ADMM framework. When the inventors derived the update equations of the fast solver in the previous section, the inventors omitted the constraints

$${W}_{i i}^{r e c} = 0 .$$

However, these constraints could be biologically relevant, and perhaps be vital to eliminate overfitting in the reproduced network. In fact, for the generator model given in Eq. 1, r_{t,i}≈r_{t+1,i} for small α; making the unconstrained problem ill-posed since self-excitation would explain a good amount of the activity of each neuron. Thus, to incorporate the equality constraint, the inventors developed a solver using the alternating direction method of multipliers (ADMM), summarized in FIG. 2.

In the ADMM solver, the primal problem is divided into two subproblems that are solved individually during separate iterations, yet linked through a consensus variable that ensures agreement between the solutions of both subproblems upon convergence. In this framework, first subproblem solves the unconstrained optimization problem whereas the second one enforces the constraints (See Appendix A for derivations). While the inventors primarily consider the equality constraint in this work, ADMM framework can be used to add additional L1 and/or nuclear norm regularizations to the learned connectivity matrix, and utilize biological priors for further inductive biases. Finally, the inventors note that given the highly irregular nature of the prediction error when d_{t,i}≈±1, the inventors supply the fast solver with a simple automated outlier detection step explained in Appendix A.

3.2 Chaotic recurrent neural network reproduction. To validate CoRNN's reproduction abilities, the inventors utilize randomly connected chaotic recurrent neural networks without any external input as generators. Specifically, the inventors pick the recurrent weights randomly from a normal distribution

$${W}_{i i}^{r e c} ∼ N ( 0 , \frac{ℊ}{\sqrt{{n}_{r e c}}} )$$

with zero mean and standard deviation of

$$\frac{ℊ}{\sqrt{{n}_{r e c}}} .$$

For g>1 anu sufficiently large n_{rec}, these networks can sustain their activities without any external stimulus and exhibit chaotic behavior. For the chaotic generators, the inventors pick g=3 throughout this disclosure to ensure that network is deep into the sustainable spontaneous activity regime. The inventors pick the chaotic regime specifically, since previous work established that teacher forcing when training RNN to output a function can have potentially harmful consequences to the stability of the network training. The inventors aim to test whether this generalizes to the reproduction task considered here.

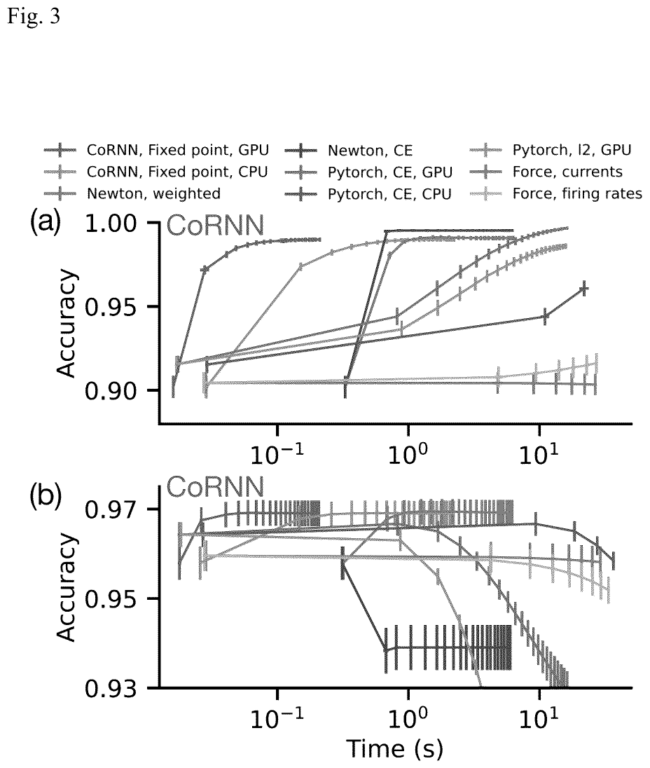

To validate that even chaotic dynamics can be reconstructed via a single step prediction error paradigm, the inventors initialized randomly connected recurrent neural networks as generators, extracted neural activities for a fixed duration of length T, and trained the dRNNs using CoRNN, Newton's solver, gradient descent, and FORCE on varying number of iterations to benchmark their speed and accuracy. All algorithms were able to train, some achieving near perfect accuracies in reproducing the internal connectivity of the generator network, as shown in FIG. 3.

Whether minimizing weighted or the naive logistic loss leads to more accurate reconstruction depended on the underlying noise distribution of the data as shown in FIG. 3, yet CoRNN outperformed all other algorithms in speed in both cases with several orders of magnitude and converged in sub-second times even with the CPU implementation, whereas others took up to several seconds for training. In fact, FORCE was not able to converge in a pre-allocated maximum training time, showing the inefficiency of FORCE training for the reproduction task. To compare, CoRNN converged faster at a given run than the computer could import the Pytorch module at the start of the code.

3.3. Robustness to regularization Having tested the speed of the algorithms, the inventors next turn their attention to robustness to regularization. If the instability claims regarding network training in generalizes to the network reproduction problem, then the single-step prediction error paradigm, regardless of the optimizer, would only be successful for a narrow range of regularization parameters.

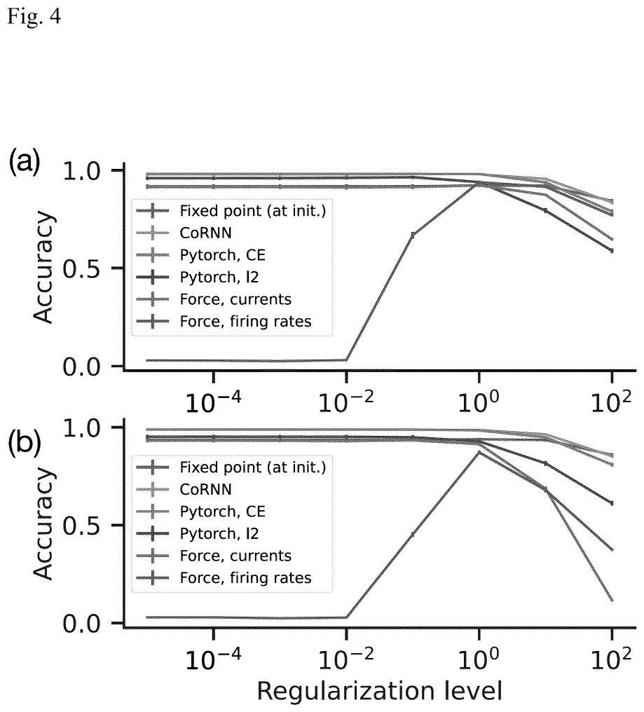

To test this scenario, the inventors first simulated a set of networks with parameters matching those in FIG. 3A and trained using various optimizers for a given number of iterations and varying (normalized) regularization levels. The results are shown in FIG. 4A, with regularization level=1 corresponding to the default regularization parameter of the respective algorithm. Unlike expectations, all single-step prediction error solvers showed high robustness to changes in the regularization level for several orders of magnitude, whereas FORCE either only maintained the accuracy achieved already the fixed-point initialization or outperformed the initialization only for a hyper-tuned regularization value and led to a completely incorrect reproduction for others. Then, the inventors tested whether the fixed-point initialization can account for the high accuracies of the single-step prediction error solvers. Specifically, the inventors initialized the networks to a random initialization instead of the fixed point and re-did the analysis of FIG. 4A in FIG. 4B. The results indicate that fixed point initialization is not necessary to obtain high accuracies, although it has two distinct advantages: i) fixed point initialization leads to a slightly higher accuracy, which the inventors test more explicitly next; and ii) it stabilizes the FORCE solver for the dRNN training. In FIG. 4B, the inventors still initialize CoRNN to the fixed point to have a reference-points between two subfigures, although CoRNN can learn without fixed-point initialization as discussed next.

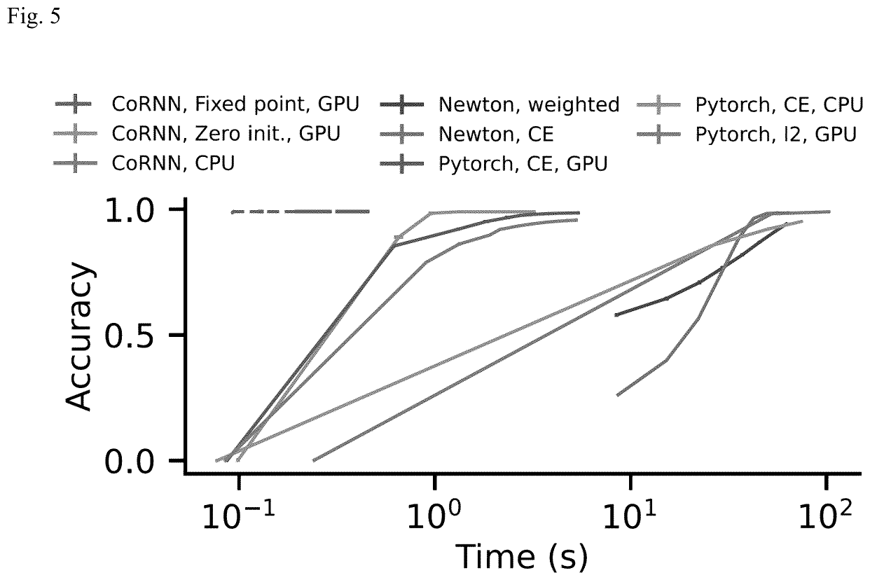

3.4. Fixed point initialization increases convergence speed. Inspired by their observation in FIG. 4B that fixed point initialization increases the accuracies slightly, the inventors next tested whether the asymptotic accuracy is increased with the better initialization, or whether the convergence speed is slowed down with the random initialization compared to fixed point initialization. To test this scenario, the inventors simulate a slightly larger network of 500 neurons, which would amplify any difference in convergence speeds compared to a smaller network, for T=10000 data points and re-ran the algorithms without fixed-point initialization. As shown in FIG. 5, CoRNN with or without fixed point initialization eventually converges to the same asymptotic accuracy, while the fixed-point initialization increases the speed of convergence at least an order of magnitude for CoRNN.

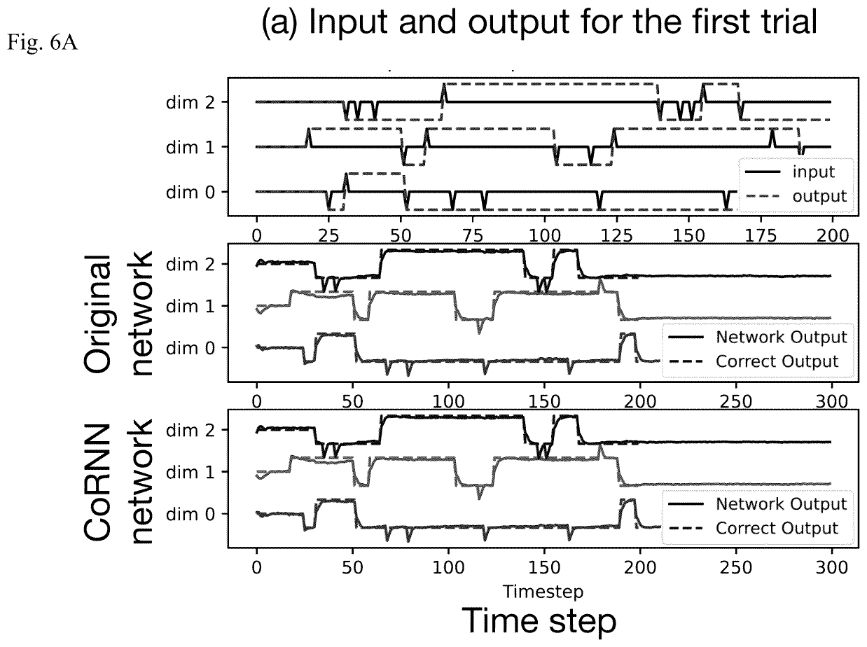

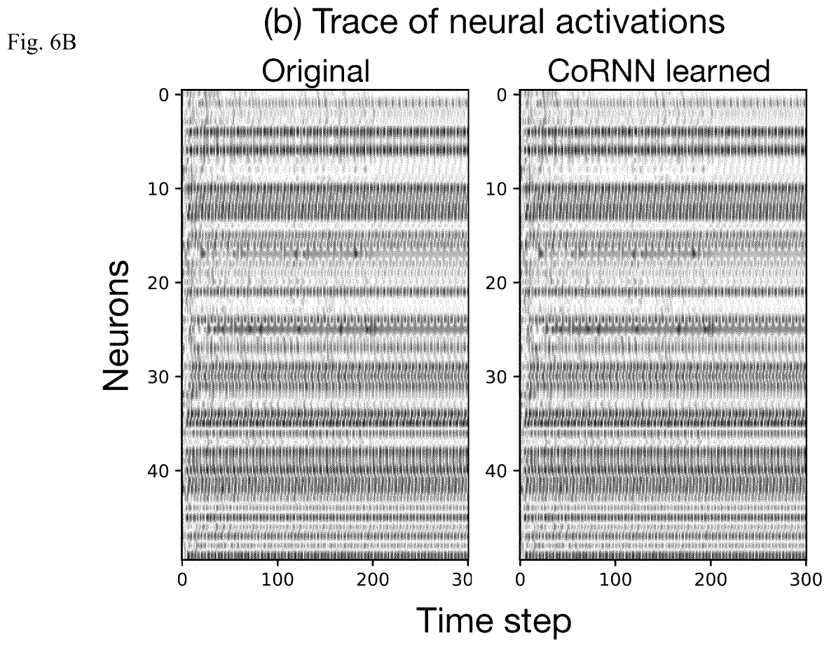

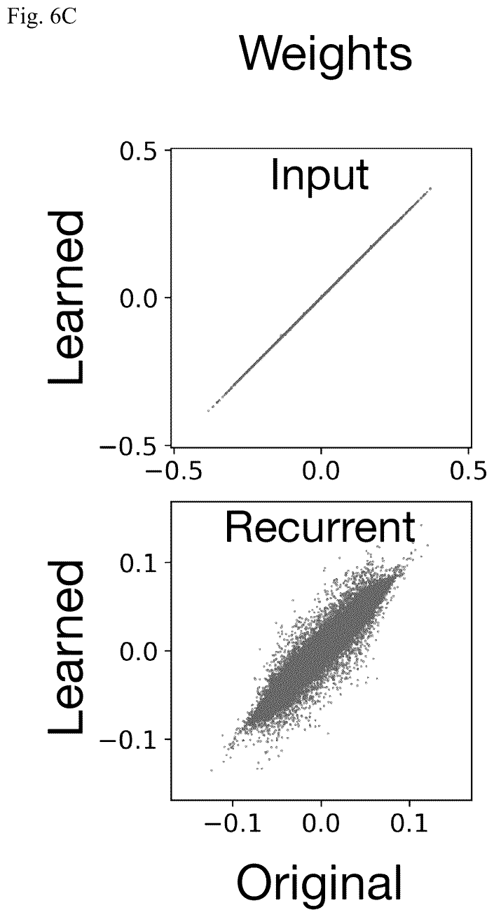

3.5. 3-bit flip flop task Having provided validity evidence for the speed and accuracy of CoRNN, or single-step prediction error solvers in general, via the randomly connected chaotic RNN generators; the inventors next focus on an RNN with a more structured connectivity. Specifically, the inventors train a model RNN to perform a 3-bit flip flop task following as shown in FIG. 6.

The 3-bit flip flop task had 3 dimensions of input and 3 of output. Each trial lasted 100 timepoints. Input dimensions could take on the values of 0, −1, or 1. At every timepoint, and for each dimension independently, there was a p=0.05 probability of being in a −1 or 1 input state with equal probability, otherwise the state was 0. The output dimensions were initialized to 0, and would “flip” to −1 or 1 depending on the input in the same dimension, and stay in that state until the input dimension took on the other value. For instance, if the first input dimension was −1 at a certain time, then the output in that dimension would be −1 from that time forward, until the first input dimension took on a value of 1. At that point the first output dimension would take on a value of 1, until the first input dimension took on a value of −1. Thus, values in the input dimensions “flip” the state of the output dimensions, like a switch.

FIG. 6A shows the output of an example test trial, not used for the generator RNN training or the CoRNN reproduction, with 200 time points followed by 100 time points of intertrial interval. Not only CoRNN reproduced network can perform the task, it gives the same output as the original network; potentially making similar mistakes as the network. Thus, as a first step, the inventors confirm that CoRNN can learn to output the same outputs as the original network.

Next, the inventors look at underlying neural activities of an example (original) generator and reproduced (CoRNN learned) network in FIG. 6B. The reproduction matches not only the output of the network, but also the internal firing dynamics of individual units. Yet, as shown in FIG. 6B, the synaptic connections are not perfectly learned even in such a simple scenario with matching models, which requires more trials and potentially interventional data as explored next. Although surprisingly, the input synaptic connections are perfectly learned in this scenario as shown in FIG. 6C, a potentially useful observation for causal biological experiments. 3.6. More trials may be necessary for larger networks Inspired by their observation that firing rates may be perfectly predicted even in test trials for large networks without exactly reproducing the underlying synaptic connectivity, the inventors next tested whether increasing the number of trials could lead to better reproduction of the original network.

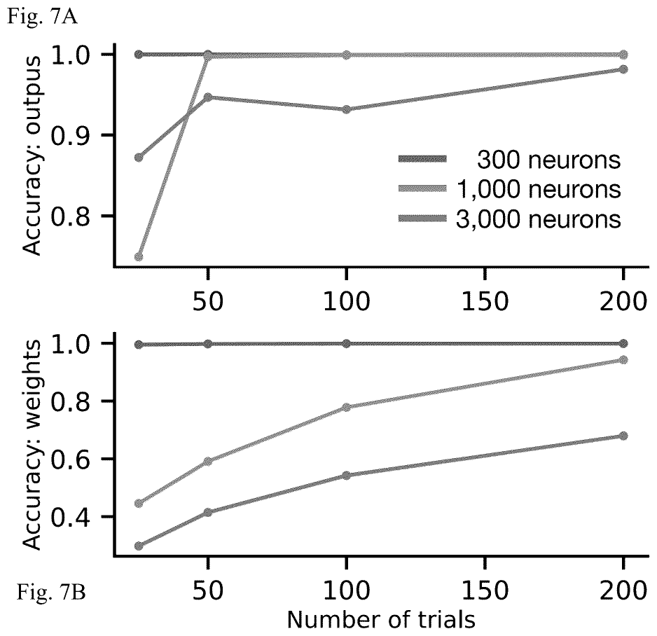

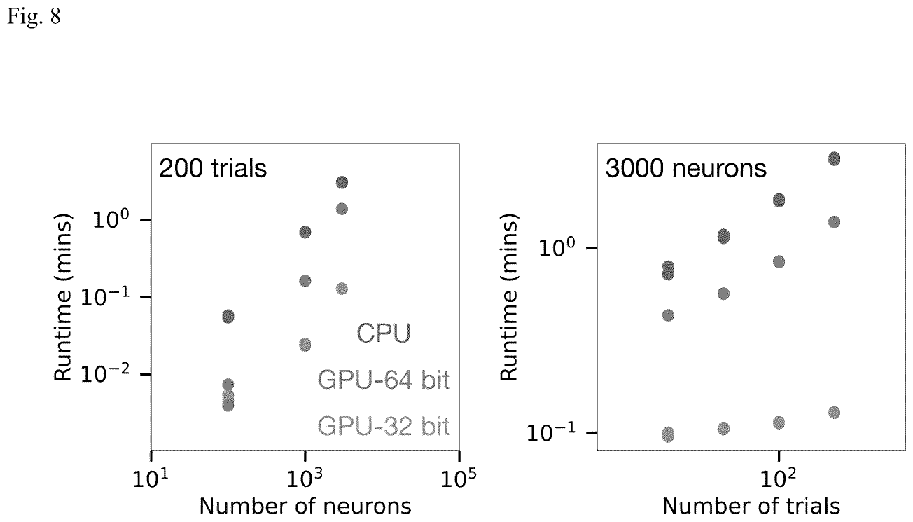

FIG. 7 shows that the output of the network can be reproduced with far smaller numbers of trials, whereas accurately reproducing the internal connectivity requires significantly more trials. The data hungriness of the estimation process increases with the number of units in the generator network, which again emphasizes the importance of an optimization algorithm that can scale well to multiple day recordings. 3.7. CoRNN scales well to larger networks and recordings So far, the inventors showed that CoRNN works faster than alternative algorithms with run times at most in few minutes. Now the inventors show that, owing to the first order nature of the updates, CoRNN also scales well to larger networks and data points as shown in FIG. 8. Remarkably, CoRNN remains sub-minute for 200 trials (approximating an hour-long recording) and 1000 neurons, meaning that CoRNN can indeed be trained real-time even in batches in biological experiments.

4. Discussion. In this disclosure, the inventors introduced a convex and scalable solver (CoRNN) for training data-constrained recurrent neural networks to reproduce brain activity. The inventors validated CoRNN on simulated data via the large-scale functional connectivity extraction task, where the inventors used both randomly connected chaotic networks and RNNs trained to perform a 3-bit flip flop task as generators. The inventors showed that CoRNN is as accurate yet faster than existing approaches by orders of magnitude and can easily scale to the large datasets of today, training in seconds to minutes depending on the number of trials and neurons. For this work, the inventors focused on developing a scalable, fast, and accurate solver for the large-scale functional connectivity extraction task. Thus, the inventors conceive several practical aspects such as subsampling of neuronal populations, miss-matches between non-linearities, miss-matches in time scales, and bringing experimental data in a suitable format for the dRNN training. Further development of data constrained RNNs can support systems neuroscience research aiming to understand interarea interactions and replace the canonical/pairwise correlations as a proxy for functional connectivity estimation. Moreover, reverse-engineering the learned network can provide deeper understanding of how neuronal populations compute. Finally, with the advent of interventional data collection methods such as single-cell targeted optogenetics, dRNNs can be trained to test causal connections computationally and supply theoretical predictions at a scale previously unachievable for experimentation.

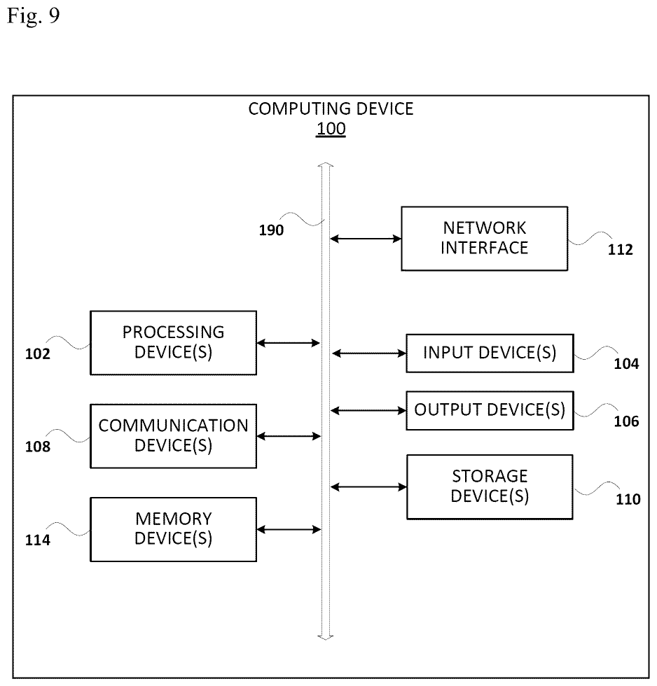

FIG. 9 is a functional block diagram illustrating an example computing device 100 configured to train a neural network to interpret brain dynamics, incorporated with teachings of the present disclosure, according to some embodiments. FIG. 9 illustrates only one particular example of computing device 100, and many other examples of computing device 100 may be used in other instances and may include a subset of the components included in the example computing device 100. Computing device 100 may include additional components not shown in FIG. 9.

As shown in the example of FIG. 9, computing device 100 includes one or more processing devices 102, one or more input devices 104, one or more output devices 106, one or more communication devices 108, one or more storage devices 110, network interface 112, and memory devices 114. Processing devices 102, input devices 104, output devices 106, communication devices 108, storage devices 110, network interface 112, and memory devices 114 are interconnected via bus 190.

Processing devices 102 may include one or more execution cores (CPUs). For example, computing device 100 may also include a peripheral controller hub (PCH) (not shown). In another example, computing device 100 may also include a sensors hub (not shown). Input devices 104 and output devices 106 may include, for example, user interface device(s) including a display, a touch-screen display, printer, keypad, keyboard, etc., sensor(s) including accelerometer, global positioning system (GPS), gyroscope, etc., communication logic, wired and/or wireless, storage device(s) including hard disk drives, solid-state drives, removable storage media, etc. I/O ports for input devices 104 and output devices 106 may be configured to transmit and/or receive commands and/or data according to one or more communications protocols. For example, one or more of the I/O ports may comply and/or be compatible with a universal serial bus (USB) protocol, peripheral component interconnect (PCI) protocol (e.g., PCI express (PCIe)), or the like.

Processing devices 102 may implement functionality and/or execute instructions within computing device 100. The instructions may be stored completely or partially within storage devices 110 and memory devices 114. For example, processing devices 102 on computing device 100 may receive and execute instructions stored by storage devices 110 that provide the functionality of training a neural network to interpret brain dynamics. These instructions executed by processing devices 102 may cause computing device 100 to store and/or modify information within storage devices 48 during program execution.

Storage devices 110 may generally comprise a random-access memory (“RAM”), a read-only memory (“ROM”), and a permanent mass storage device, such as a disk drive or SDRAM (synchronous dynamic random-access memory). Computing device 100 may store program code for modules and/or software routines.

Storage devices 110 may also store operating system, kernel, kernel space, user space, and user protected address space. Storage devices 110 may store one or more processes (i.e., executing software application(s)). One or more processes may generally execute in parallel, i.e., as a plurality of processes and/or a plurality of threads. These software components may be loaded from a non-transient computer-readable storage medium into storage devices 110 using a drive mechanism associated with a non-transient computer-readable storage medium, such as a floppy disc, tape, DVD/CD-ROM drive, memory card, or other like a storage medium. In some embodiments, software components may also or instead be loaded via a mechanism other than a drive mechanism and computer-readable storage medium (e.g., via network interface 112).

Embodiments of the presently disclosed invention are a computer software system and a solution to an important optimization problem. Some embodiments include convex optimization method that fits a recurrent neural network to reproduce the neuronal activities of mice or other animals observed from experimental data through calcium and voltage imaging. Some embodiments could fall within the domain of “listening to” the neural activities, but beyond listening, embodiments model the activities. Moreover, some embodiments have practical implications for brain machine interface research.

A distinguishing factor of some embodiments of the presently disclosed invention to previous technology is the formulation of the problem in a language that matches the biological reality of experiments. FORCE, the previous approach, has several short-comings that is described herein. Moreover, the inventors develop the convex solver that is 1-2 order of magnitude faster than the closest competition.

Among many possible uses or applications of some embodiments of the presently disclosed invention are brain-machine interfaces using this approach, research regarding large-scale connectivity/discovery of functionally connected brain circuits from calcium imaging recordings etc. Embodiments of the presently disclosed invention may be used with efferent BMIs, passive BMIs, sensory BMIs, two-photon (2P) calcium imaging, genetically encoded calcium indicators (GECOs), for example.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. The present disclosure also contemplates other embodiments “comprising,” “consisting of” and “consisting essentially of,” the embodiments or elements presented herein, whether explicitly set forth or not. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items, while only the terms “consisting of” and “consisting only of” are to be construed in the limitative sense.

1. A computer-implemented method for training a neural network to interpret neural dynamics, the method comprising:
obtaining functional connectivity between brain regions and artificial neurons simulated by the neural network;
capturing neural activities between biological neurons;
processing the captured neural activities to obtain training data for the neural network; and
constructing the neural network using the training data to reproduce neural dynamics between the artificial neurons.

2. The method of claim 1, wherein the neural network includes a convex solver.

3. The method of claim 2, wherein the convex solver comprises a convex weighted loss function.

4. The method of claim 2, further comprising optimizing a loss of the convex solver via a fixed-point estimation.

5. The method of claim 1, further comprising predicting a spatial and temporal activity of neural population dynamics.

6. The method of claim 1, wherein the neural network is configured to extract large-scale functional connectivity between brain regions and/or simulated networks.

7. The method of claim 1, wherein the neural network is trained to predict the temporal activity of a corresponding biological neuron in the brain.

8. The method of claim 1, wherein the neural network is configured to uncover attractor structures within the brain dynamics.

9. The method of claim 1, wherein the neural network is further configured to design external interventions to control neural population dynamics in the brain.

10. The method of claim 1, wherein the neural network is trained to interpret neural dynamics in real-time to support brain-machine interfaces.

11. The method of claim 2, wherein the convex solver is further configured to perform optimization updates accelerated on a graphics processing unit (GPU).

12. The method of claim 2, wherein the convex solver is part of an alternating direction method of multipliers (ADMM) framework.

13. The method of claim 2, wherein the convex solver is configured to enforce constraints on the neural network during training.

14. The method of claim 13, wherein the constraints include preventing self-excitation of neurons within the neural network.

15. The method of claim 2, wherein the convex solver is configured to utilize biological priors for inductive biases during training.

16. The method of claim 2 any one of claims 2-4 and 11-15, wherein the convex solver is configured to handle input noise and conversion noise during the training of the neural network.

17. The method of claim 2 any one of claims 2-4 and 11-16, wherein the convex solver is configured to perform outlier detection during the training process.

18. The method of claim 2 any one of claims 2-4 and 11-17, wherein the convex solver is configured to scale to datasets collected from more than 10,000 neurons for offline processing.

19. A non-transitory computer-readable medium comprising instructions that, when executed by one or more processors, cause a computer system to perform the method of claim 1.

20. A computer system for training a data-constrained recurrent neural network to interpret neural dynamics, the system comprising:
one or more processors; and
a non-transitory computer-readable medium including instructions that, when executed by the one or more processors, cause the computer system to perform the method of claim 1.

21. (canceled)
